(19) United States
(12) Patent Application Publication
(10) Pub. No.: US 2023/0226349 A1
(43) Pub. Date: Jul. 20, 2023

(54) SUBDERMAL NEEDLE ELECTRODE APPARATUS FOR BIOFILM INFECTION CONTROL

(71) Applicant: GARWOOD MEDICAL DEVICES, LLC, Buffalo, NY (US)

(72) Inventor: Timothy M. Kopera, Lancaster, NY (US)

(73) Assignee: GARWOOD MEDICAL DEVICES, LLC, Buffalo, NY (US)

(21) Appl. No.: 17/928,974

(22) PCT Filed: Jun. 5, 2020
(86) PCT No.: PCT/US2020/036270

§ 371 Date: Dec. 1, 2022

(51) Int. Cl. A61N 1/20; A61M 5/142

(52) U.S. Cl. CPC A61N 1/20; A61M 5/14276

(57) ABSTRACT

An apparatus for use with a needle electrode assembly used to disrupt biofilm from an implanted metallic object, such as a replacement orthopedic appliance, includes a body that retains the needle electrode assembly including a projecting needle portion projecting from a lower end of the body. A needle adjustment mechanism enables the length of the projecting needle portion to be adjusted, the needle adjustment mechanism including a spring element within the apparatus body and attached to the needle electrode assembly. The spring element has a substantially nonvariable spring constant such that constant pressure is applied to the needle electrode irrespective of the length of the needle electrode to be brought into contact with the skin of a subject and implanted metallic object, once released by the needle adjustment mechanism. The apparatus includes a plurality of supports that enable the apparatus to be attached to the skin of a subject during treatment wherein a retraction mechanism enables the needle to be withdrawn into the apparatus and a locking mechanism enables safe encapsulation and retention of the needle within the apparatus after treatment.

### TECHNICAL FIELD

This application is generally directed to the treatment of metallic objects and more specifically directed to an apparatus used to support and/or position a needle electrode for treatment and disruption/removal of biofilms caused by bacterial infection to metallic objects, such as orthopedic implants.

### BACKGROUND

With the number of medical advancements being made, surgically implanted orthopedic devices such as knee and shoulder replacements, have become increasingly common for individuals. A prevailing issue in regard to these implanted devices is infection. A number of devices have been created for the removal of biofilm and bacterial treatment from metallic implants, including various treatment apparatus that have been developed by Applicant, as opposed to having to surgically remove and replace the implanted device. In terms of operation, a needle electrode is inserted into the patient for contacting the metallic implant while another electrode is attached to the skin of the patient. Each of the electrodes are connected to a device that creates an electrical current via an electrochemical reaction.

These treatment systems have been highly effective. However, there are still opportunities to improve their design and efficacy, as well as being able to conduct treatments repeatably with regard to disparate classes of patients of differing sizes.

There is a further safety and environmental concerns since the needle electrode used in biofilm treatment is placed in direct contact with the metal implant and therefore is covered with potentially toxic material(s). Accordingly, there is a need to be able to withdraw and maintain the needle electrode safely and reliably, without fear of contamination to others.

### BRIEF DESCRIPTION

Therefore and according to at least one aspect, there is provided an apparatus for either positioning and/or supporting a needle electrode used to disrupt biofilm from an implanted metallic object. The apparatus includes a body defining a cavity configured to retain a needle electrode assembly, the needle electrode assembly including a needle extending from the body at one end and an opposite end that is couplable to an electrical source. A needle adjustment mechanism enables the length of the needle extending from the body to be adjusted relative to a subject having the implanted metallic object, the needle adjustment mechanism comprising a spring element disposed within the body and in contact with the needle electrode assembly, wherein the spring element is a constant force spring element that maintains constant pressure on the needle irrespective of the length extending from the body.

In at least one embodiment, the apparatus can include a cap that covers the extending portion of the needle electrode initially prior to use. In at least one version, the cap can be threadingly engaged with one end of the body. The needle adjustment mechanism is configured to initially release the needle electrode and enable the extending length of the needle electrode to be selectively varied for placement in contact into the skin of the subject and more specifically the metal implant. In one version, a release pin accessible to the user can be opened to permit the extending length of the needle electrode to be selectively varied with pressure being applied by the spring element. Because the spring element has a substantially nonvariable spring constant, constant pressure is applied to the implant irrespective of the length of the extending needle electrode.

According to this specific embodiment, the apparatus further comprises a plurality of support legs that are hingably attached to the outer surface of the body, each of the support legs having a support foot that is shaped and configured to be affixed to the skin area of a patient/subject. The support feet are connected to ends of the support legs using ball joints or similar structure and preferably include an adhesive pad to be placed in contact with the skin of the subject. Preferably, each of the support feet are designed to be sufficiently flexible to conform to the various contours of the subject.

Therefore, the needle adjustment mechanism of the herein described apparatus maintains the needle electrode fully exposed to facilitate penetrating the skin of the subject and finding the metal implant. Following release, the length of the exposed needle can be varied with the constant force spring element adding positive pressure irrespective of the length of the exposed portion of the needle electrode to maintain contact with the metal implant. The support legs and feet are then utilized to secure the apparatus to the subject during treatment.

The herein described apparatus further comprises a retraction mechanism, which can be selectively activated following treatment in order to withdraw the needle electrode from the patient and into the body of the apparatus, while an additional locking mechanism is configured to effectively lock the needle electrode in an encapsulated state for safe disposal. The locking mechanism provides a mechanical stop for the released portion of the apparatus, while also preventing the needle electrode from being re-engaged or redeployed from the body of the apparatus.

According to another aspect, there is provided a method for treating an implanted metallic object, the method comprising supporting a needle electrode assembly within an apparatus body, the needle electrode including a needle having an exposed length extending from the apparatus body at one end and connected at an opposing end to an electrical source. The exposed length of the needle can be adjusted relative to a subject having the implanted metallic object, the needle electrode assembly being attached to a spring element supported within the body. Using the needle electrode, biofilm from the implanted metallic object can be disrupted, and then according to the method, retracting and retaining the exposed portion of the needle and needle electrode assembly within the body of the apparatus for disposal.

An advantage provided by the herein described apparatus is that a single needle electrode can be used for treatment irrespective of the size and physical condition of the patient, with a constant pressure being applied to the needle electrode, regardless of the extending length of the needle electrode.

Another advantage provided is that the herein described treatment apparatus can safely and securely hold an electrified needle electrode against a metallic object, such as an orthopedic implant, with constant positive pressure for an extended period of time. The apparatus is further advantageously configured to enable safe disposal of the needle electrode following treatment. Moreover, the herein described apparatus is further configured to prevent re-use of a needle electrode following treatment of a subject/patient.

These and other features and advantages will be described in greater detail with reference to the following Detailed Description, which should be read in conjunction with the accompanying drawings.

### BRIEF DESCRIPTION OF THE DRAWINGS

FIG. 1 is a perspective view of an apparatus made in accordance with aspects of the present invention;

FIG. 2(a) is a partial side elevational view, taken in section, of the apparatus of FIG. 1;

FIG. 2(b) is the partial side elevational view of the apparatus of FIG. 2(a), the view being rotated 90 degrees about the vertical axis;

FIG. 3(a) is an exploded assembly view of the apparatus of FIGS. 1-2(b);

FIG. 3(b) is a top perspective view of the holder base of the supporting and positioning apparatus of FIGS. 1-3(a);

FIG. 3(c) is a bottom perspective view of the holder cover of the supporting and positioning apparatus of FIGS. 1-3(a);

FIG. 4 is a perspective view of a needle electrode assembly used in the apparatus of FIGS. 1-3;

FIG. 5 is an enlarged sectioned view of a portion of the needle electrode assembly of FIG. 4;

FIG. 6 is partial side elevational view in section of a portion of the apparatus of FIGS. 1-3, showing a release feature for the retained needle electrode assembly in accordance with aspects of the invention;

FIG. 7 is a partial side elevational view of a portion of the apparatus of FIGS. 1-3 and 6, depicting the removal of a protective cap in accordance with aspects of the invention;

FIG. 8 is a perspective view of the apparatus of FIGS. 1-3, 6 and 7 showing the exposed needle of the needle electrode assembly of FIGS. 4 and 5 and the removed protective cap;

FIG. 9 is a partial sectioned view of the apparatus of FIGS. 1-3 with the release feature still attached;

FIG. 10 is the partial sectioned view of the apparatus and more specifically action being taken on the release feature;

FIG. 11 is the partial sectioned view of the apparatus according to FIGS. 7-9, and following the action taken on the release feature, now enabling movement of the needle electrode assembly;

FIGS. 12, 13 and 14 are partial sectioned views of the apparatus in accordance with aspects of the invention, and more specifically depicting various extending lengths of the needle electrode assembly against a contained spring element;

FIG. 15 is a perspective view of a constant force spring element used in the apparatus in accordance with aspects of the invention;

FIG. 16 is a sectioned view of the apparatus, further depicting pivotal movement/deployment of the support legs;

FIG. 17(a) is a sectioned view of the apparatus of FIG. 16, rotated by 90 degrees about the vertical axis and further depicting the support feet of the apparatus;

FIG. 17(b) is a perspective view of the apparatus of FIG. 17(a) with the support legs in the deployed position;

FIG. 18 is an enlarged perspective view illustrating the pivotal attachment of a support foot to the end of a support leg of the apparatus;

FIG. 19 is a top perspective view of a support foot of the apparatus in accordance with aspects of the present invention;

FIG. 20 is a partial sectioned view of the top portion of the apparatus of FIGS. 1-3;

FIG. 21 is the partial sectioned view of FIG. 20, depicting portions of the release features of the apparatus;

FIG. 22 is a top plan view of the apparatus of FIGS. 20 and 21;

FIG. 23 is a partial sectioned view of the bottom of the apparatus following engagement of a needle retraction mechanism;

FIGS. 24 and 25 depict the partial sectioned view of the apparatus of FIG. 23, depicting progressive retraction of the needle electrode into the body of the apparatus using the needle retraction mechanism;

FIG. 26 is a partial sectioned view of the apparatus depicting the action of a locking mechanism to prevent over advancement of the needle electrode assembly within the body of the apparatus;

FIG. 27 is the partial sectioned view of FIG. 26 of the apparatus, depicting various features of the locking mechanism that prevent the needle electrode from being redeployed; and

FIG. 28 is a front perspective view of a portion of the locking mechanism in accordance with aspects of the present invention.

### DETAILED DESCRIPTION

The following description relates to an exemplary embodiment of an apparatus used to either position and/or support a needle electrode used in treatments that provide electrical disruption of bacterial biofilms that have accumulated over time onto the surface of a metal surgically implanted orthopedic device, such as a knee replacement. It will be readily understood that the herein described apparatus can be used in connection with other implanted metal orthopedic devices. In addition, a number of terms are used throughout this discussion in order to provide an adequate frame of reference with regard to the accompanying drawings. These terms, which include “inner”, “outer”, “internal”, “external”, “above”, “below”, “distal” and “proximal”, among others, should not be interpreted narrowly except where so specifically noted throughout the discussion. Still further, the accompanying drawings are intended to convey the salient features of the invention in which several figures have been enlarged to better aid their discussion. Accordingly, the figures should not be necessarily relied upon for scaling purposes.

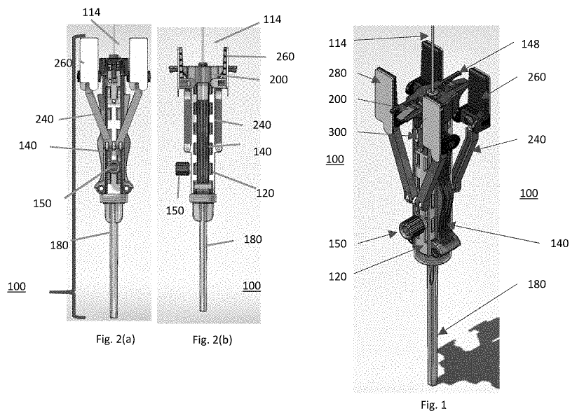

With reference to FIGS. 1-3(a), the main components of the apparatus 100 are shown in accordance with the exemplary embodiment. These components include an apparatus body or holder primarily made from a pair of interconnected body components, namely a holder base 120 and a holder cover 140. As shown in greater detail in FIGS. 1— 3(b), the holder base 120 is an elongate member made from a structural material, such as a moldable plastic, that includes a pair of parallel and spaced sidewalls 124 that are supported at a lower end 125. The sidewalls 124 create a spacing therebetween over the length of the holder base 120 between the lower end 125 and an upper end 123. Each of the sidewalls 124 further include a pair of spaced interior slots 127 extending from the open upper end 123 and terminating just above the lower end 125 along the entire length of each sidewall 124 of the holder base 120. The lower end 125 of the holder base 120 according to this specific embodiment is defined by a cylindrical configuration (although other shapes can be used) and is closed with the exception of a center opening 128, FIG. 6, the latter opening 128 being sized and configured to receive the exposed portion of a retained needle 108 of a needle electrode assembly 104.

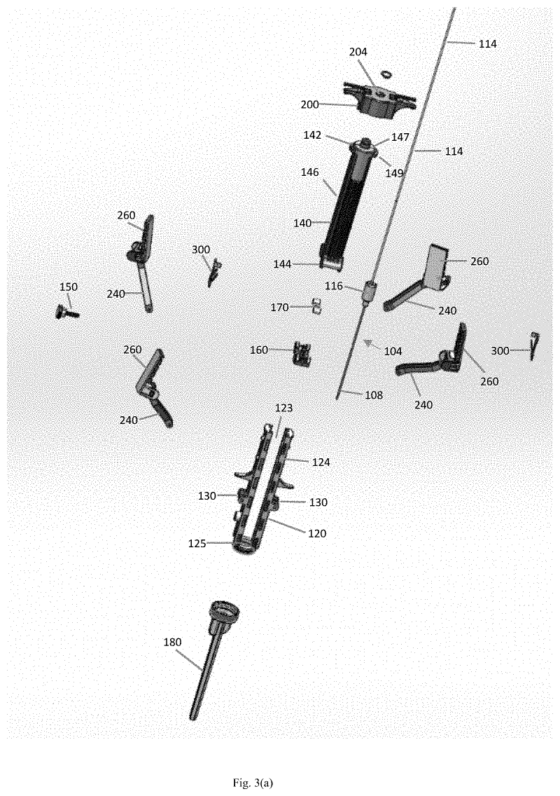

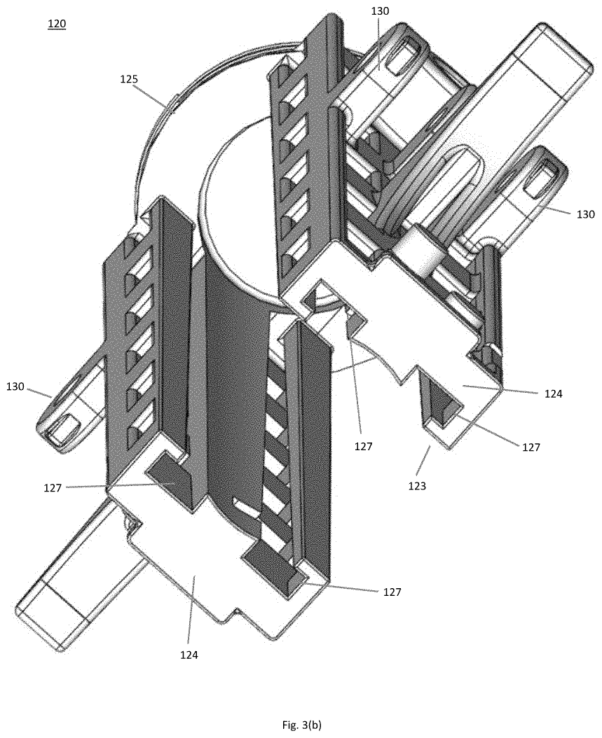

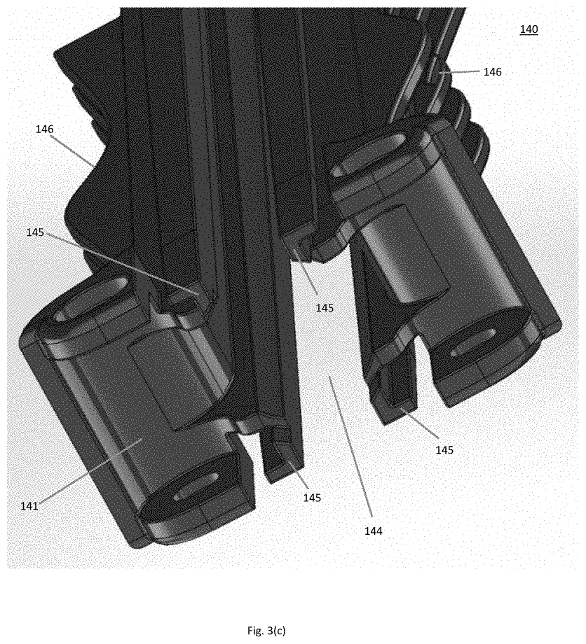

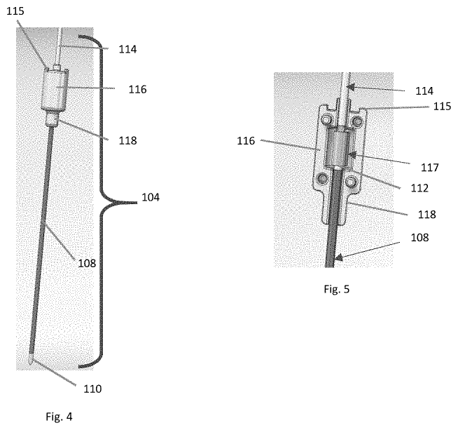

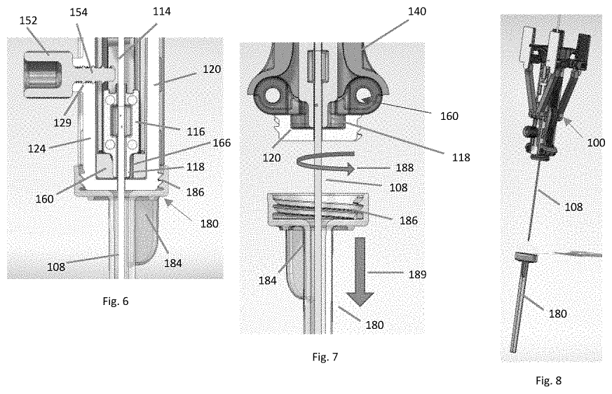

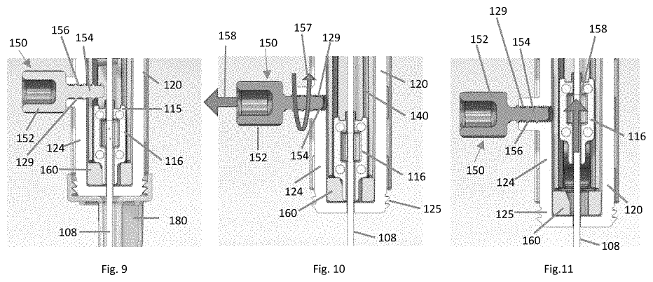

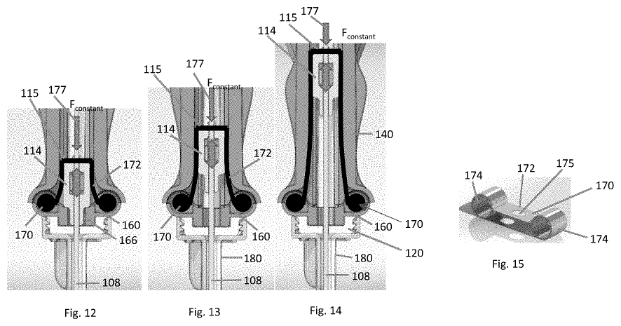

The holder cover 140, which is shown in FIGS. 1-2(b), 3(a) and 3(c), is also an elongate member that includes a closed top end or portion 142 and an open bottom end 144. A pair of parallel sidewalls 146 are provided between the top and bottom ends 142, 144 having a spacing therebetween. Each of the sidewalls 146 includes a set of vertical rails 145 extending between the top and bottom ends 142, 144 that are configured to engage the interior slots 127 of the holder base 120. As such, the holder cover 140 is movable relative to the holder base 120. When assembled as shown in FIGS. 1-2(b), the sidewalls 124 of the holder base 120 are orthogonal to the sidewalls 146 of the holder cover 140, thereby creating the body of the herein described apparatus 100. The top portion 142 of the holder cover 140 is defined, according to this embodiment by a substantially cylindrical configuration, including a set of projecting tabs 149 on opposing sides of the top portion 142, as well as an upper projecting portion 147, the latter projecting portion 147 having a center through opening 148, FIG. 1, which is aligned axially with the center opening 128, FIG. 6, of the holder base 120, when the body/holder is fully assembled. The projecting tabs 149 are aligned with the top of the sidewalls 124 of the holder base 120.

Still referring to FIGS. 1-3(a), a retaining cap 200 is releasably attached to the top portion 142 of the holder cover 140. The retaining cap 200 according to this specific embodiment is defined by a hollow cylindrical member that is sized and configured to cover the top portions 123, 142 of the holder base 120 and the holder cover 140, respectively. As discussed in greater detail and according to this embodiment, the retaining cap 200 includes at least one release feature on opposing sides that engage the projecting tabs 149 of the holder cover 140, as well as a top ledge 121, FIG. 20, of the sidewalls 124 of the holder base 120, as described in greater detail below. The retaining cap 200 also includes a center opening 204 that is axially aligned with the openings 128 and 148 of the holder base 120 and the holder cover 140 and receives the upper projecting portion 147.

A set of support leg mounts 130 are intermediately disposed on the exterior surface of each of the sidewalls 124 of the holder base 120 between the upper and lower portions 123, 125. According to this embodiment, each set of support leg mounts 130 are shaped and configured to support a pair of support legs 240 on opposing sides of the holder base 120. Each support leg 240 comprises a support foot 260 at one end, wherein each of the support legs 240 are pivotally mounted to the holder base 120 at an opposing end via the set of support leg mounts 130. Preferably and according to this embodiment, the support legs 240 are configured for movement between a storage position, see FIG. 8, and a deployed position, see FIG. 17(b), as discussed herein in greater detail.

As shown in FIG. 3(a), the open lower end 144 of the holder cover 140 includes a bottom recessed portion 141, FIG. 3(c), that is sized and configured to retain a needle saddle 160, the latter being designed to retain a spring element 170 used as part of the needle electrode assembly 104. According to the herein described embodiment, the needle electrode assembly 104 is configured for movement within a cavity in the defined apparatus body defined by the holder base 120 and the holder cover 140. The cavity or pathway creates a self-contained, hollow structure for the needle electrode assembly 104 to travel, as further discussed in detail.

Still referring to FIGS. 1— 3(a), the herein described apparatus 100 further includes a needle release member 150, such as a release knob, as well as a locking mechanism 300. As discussed in greater detail below, the release member 150 is configured to extend into the pathway defined by the body of the apparatus and initially restrict movement of the needle electrode assembly 104 with the entire length of the projecting portion 108 being exposed. Upon release of the needle release member 150 from the body of the apparatus 200, the length of the projecting portion 108 can be selectively adjusted relative to a subject in advance of treatment with the support legs 240 being configured for attachment to the skin area of the subject. The needle electrode assembly 104 is configured to be fully withdrawn from a subject and into the body of the apparatus 100 following treatment and the locking mechanism 300, as further discussed herein, prevents the needle electrode assembly 104 from downward movement and inadvertent redeployment following treatment and withdrawal. In addition, a protective cap 180 covers the exposed portion 108 of the needle electrode assembly 104 prior to use. Each of the above features of the supporting and positioning apparatus 100 are now further described in greater detail.

First and with reference to FIGS. 4 and 5, the needle electrode assembly 104 according to this specific embodiment is comprised of a needle 108, the latter being a thin elongate member made from steel or other electrically conductive material and having an electrically insulative coating over the majority of its length with each of the opposing distal and proximal ends 110, 112 of the needle 108 being left uncoated. An electrode wire 114 is attached to the proximal end 112 of the needle 108 in such a way that an electrical connection 117 can be made. This electrical connection 117 could include any suitable technique, such as soldering or crimping a splice over the two engaged elements 104, 114.

The electrode wire 114 outwardly extends from the apparatus 100, as shown in FIG. 1, through the center opening 148 in the upper portion of the holder cover 140 and is configured to make an electrical connection with a device such as a potentiostat (not shown), which is responsible for generating a suitable amount of electrical current used for the treatment of the metallic implant. The above-noted process of electrical stimulation and an exemplary treatment system is described in further detail in U.S. Pat. No. 9,616,142, the entire contents being incorporated herein by reference.

The electrical connection 117 (solder, crimp, etc.) between the needle electrode 104 and the electrode wire 114 according to this embodiment is encapsulated by a housing 116. The housing 116 according to this embodiment is polymer-based, but can be made from any suitable electrically insulative material. The housing 116 of the needle electrode assembly 104 is defined by a constant diameter over the majority of its length, with the exception of a lower projecting portion 118, this latter portion 118 having a substantially narrowed diameter.

According to this embodiment and with reference to FIG. 3(a), a needle adjustment mechanism of the apparatus 100 further includes the spring element 170, the latter being supported by the needle saddle 160 within the lower recess 141 of the holder cover 140. More specifically, the needle saddle 160 is defined by a unitary body made from a structural material having recesses formed on opposing sides, each recess being sized and configured to support the spring element 170, as well as a center opening 166, FIG. 6, that is sized to permit the passage of the extending needle 108 as well as the lower projecting portion 118 of the housing 114.

When assembled, and as shown in FIG. 6, the needle electrode assembly 104 is initially disposed within the cavity defined within the holder base 120 and holder cover 140. More specifically, the lower projecting portion 118 of the housing 116 is initially positioned within the center opening 166 of the needle saddle 160, the latter being secured at the lower end 144 of the holder cover 140, with the needle 108 being fully exposed and extending through the lower end 125 of the holder base 120. The housing 114 of the needle electrode assembly 104 is in direct contact with the needle saddle 160, since the center opening 166 of the needle saddle 160 is sized only to permit the passage of the lower projecting portion 118.

With reference to FIGS. 6-8, and to prevent any unintentional needle sticks, the herein described supporting and positioning apparatus 100 further includes the protective cap 180. The protective cap 180 attaches to the lower end 125 of the holder base 120 and covers the exposed portion of the needle 108, which outwardly extends through the center opening 129 formed in the lower end 125 of the holder base 120. According to this specific embodiment, the protective cap 180 is an elongate member made from a moldable plastic or other suitable structural material having an internal cavity 182 that is shaped and sized to retain the fully exposed length of the extending needle 108. According to this specific embodiment, the protective cap 180 includes an upper grippable portion 184 having a set of internal threads 186 configured for engagement with a corresponding set of external threads 126 that are provided at the lower end 125 of the holder base 120. As such, the protective cap 180 is enabled for releasable attachment to and removal, see arrow 189, from the body of the supporting and positioning apparatus 100, as shown in FIGS. 7 and 8. Other suitable methods can be used for securing and releasing the protective cap from the apparatus body in lieu of a threaded connection. For example, an interference fit can be created between the protective cap and the lower end of the holder base 120, without necessarily requiring any rotation of the protective cap 180.

As shown in FIGS. 9-11, the needle electrode assembly 104 is initially prevented from upward movement within the pathway defined by the holder base 120 by the needle release member 150, the latter feature extending into the pathway defined within the holder base 120 and forming part of the needle adjustment mechanism. According to this specific embodiment, the needle release member 150 is defined by a head portion 152 and an extending shank portion 154. The shank portion 154 has a smaller diameter than the head portion 152 and is further configured with a set of threads 156 configured to engage a threaded opening 129 in a projecting portion 122 formed in one of the side walls 124 of the holder base 120, adjacent the lower end 125. According to this embodiment, the threaded opening 129 further extends into the sidewall 124.

While attached, the shank portion 154 of the needle release member 150 extends into the pathway of the holder base 120 immediately above the upper portion 115 of the housing 116 of the needle electrode assembly 104. The needle release member 154 initially prevents upward movement of the needle electrode assembly 104 and permits the fully exposed needle 108 to be accessible. In operation and once the needle 108 has been inserted into the patient and the user is satisfied with its placement, the head portion 154 of the needle release member 150 can be unscrewed (e.g., in a counterclockwise direction as shown by arrow 157) and withdrawn (per arrow 158), as shown in FIG. 10. Unscrewing the needle release member 150 moves the shank portion 154 from the defined cavity or pathway of the holder base 120 and permits unobstructed travel of the needle electrode assembly 104. This movement advantageously allows for one needle length to be utilized for several different patient “sizes” and does not require the user to detect a predetermined needle length required for each treatment or procedure. By allowing the needle electrode assembly 104 to slide into the lower end 125 of the holder base 120, the apparatus 100 may be brought in contact with the patient's skin.

As shown in FIGS. 12-14, the spring element 170 is secured about the exterior of the housing 116 of the needle electrode assembly 104, and more specifically about the upper end 115. This arrangement enables downward pressure to be applied to the extending needle 108 during treatment during which the uncoated distal end 110, FIG. 4, is directly engaged with a metallic implant (not shown). According to this embodiment and as shown in FIG. 15, the spring element 170 is preferably a constant force spring element defined by a ribbon of spring material 172 disposed about a pair of spools or drums 174 on opposing sides and extending therein for engagement with the needle electrode assembly 104, the ribbon including an opening 175 that permits direct attachment to the upper section 115 of the housing 116. Preferably and according to this specific embodiment, the spring element 170 used does not have a spring constant that substantially varies with the amount of extension. Various spring extensions are illustrated in FIGS. 12-14, see arrows 177 for spring extensions of 0.5 inches, 1.5 inches and 3.0 inches, respectively, based on upward movement and retraction of the exposed needle 108 of the needle electrode assembly 104. Unlike linear springs such as extension springs, a constant force spring element, such as those made by Vulcan Springs, among others, is configured to apply roughly the same amount of force onto the secured needle 108, independent of the extension the ribbon 172 of the spring element 170 has traveled (i.e., Hooke's law does not apply).

Accordingly and in a resting/initial condition in which no exterior forces are applied to the needle 108, the constant force spring element 170 holds the needle 108 firmly against the needle saddle 160. As previously discussed, the needle saddle 160, retains the spring element 170 and is supported within the lower end 143 of the holder cover 140.

Therefore and once the needle release member 150 is withdrawn from the body of the apparatus 100, as discussed herein, the length of the exposed needle 108 can be suitably adjusted depending on the subject (patient) in advance of treatment. The needle electrode assembly 104 is movable within the apparatus 100 against the bias of the contained constant force spring element 170, the latter being secured about the upper section 115 of the housing 116.

Alternatively, other means can be provided in lieu of the constant force spring element using mechanical, pneumatic or other forces each configured to provide positive pressure of the supported needle electrode. The inclusion of positive pressure not only enables the length of the needle electrode to be suitably adjusted depending on the patient, but also insures that the electrode reliably maintains pressure against the implant at all times during treatment.

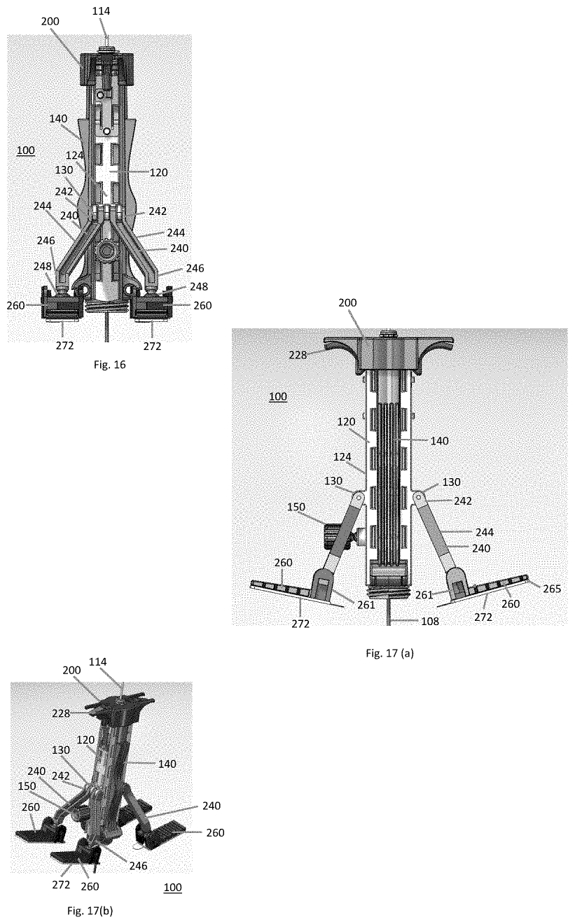

With reference to FIGS. 16, 17(a) and 17(b), the support legs 240 of the herein described apparatus 100 can be moved from a stored position, as shown in FIG. 8, to a deployed position for attachment to the skin area of a subject/patient. According to this embodiment, the support legs 240 are defined by a holder base mount end 242 having a spacing that is configured to be fitted about at least one of the support leg mounts 130 formed on the sidewalls 124 of the holder base 120. Once fitted thereon, a threaded fastener (not shown) can be disposed through transverse and aligned openings formed in the set of support leg mounts 130 and the holder base mount end 242 of a pair of attached legs 240.

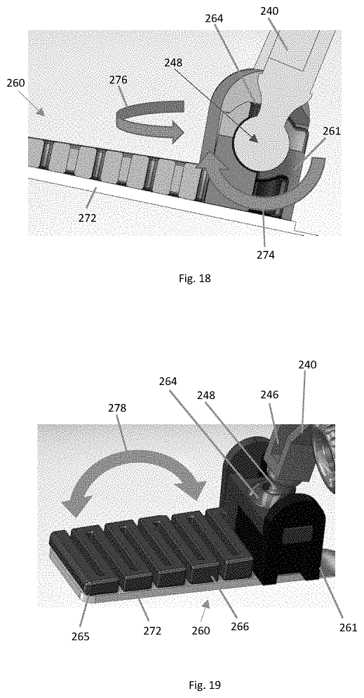

According to this embodiment, each support leg 240 further includes an intermediate leg portion 244 disposed between the holder base mount end 242 and an opposite support foot mount end 246, the latter end 246 including a ball joint 248 configured for engagement within an opening 264 formed in the proximal end 261 of a support foot 260. With further reference to FIGS. 18 and 19, the support feet 260 are further defined by a base 266 made from a flexible molded plastic and preferably formed in a serpentine configuration defined substantially by an “S-shape” between the proximal end 261 and a distal end 265 of each support foot 260. The serpentine pattern formed according to this embodiment provides considerable flexibility for each support foot 260, as opposed to a fully solid section. According to this embodiment, an adhesive pad 272 is attached to an underside of the base 266 of each support foot 260, the latter enabling attachment to the skin of a patient (not shown).

According to this specific embodiment, each of the support legs 240 can be moved from the stored position shown in FIG. 8 to a deployed position, shown in FIG. 17(b), based on the pivotable connection of the support legs 240 with the holder base 120. More specifically, the support legs 240 are connected to the set of support leg mounts 130 of the holder base 120 in a manner such that they are allowed to pivot about their attachment point by 180 degrees between the stored and deployed positions. In addition and with regard to the attachment of the support feet 260, the ball joint 248 of each support leg 240 allows for maximum rotational freedom as shown by arrows 274, 276 and 278 to help contour the support feet 260, especially given the diverse spectrum of patient shapes and sizes that may be typically encountered. As noted, the support feet 260 are also designed to have inherent flexibility in order to aid in the contouring process. The flexibility is achieved by the fabricated serpentine configuration of the base 266 of each support foot 260. When the support legs 240 are in the deployed position, the adhesive pads 272 of the support feet 260 can be brought into contact with the patient's skin (not shown). In operation and once each of the adhesive pads 272 of the support feet 260 are adhered in place to the skin of the patient, the positive pressure maintained by the constant force spring element 170 on the needle electrode assembly 104 will be capable of handling any disturbances during biofilm removal and maintain contact of the extending portion of the needle 108 with the metallic implant (not shown), thereby allowing for uninterrupted treatment of the subject.

The process for conducting a treatment can be undertaken with the extending needle 108 of the needle electrode assembly 104 at the desired length and with an electrical connection being made between the electrode wire 114 and potentiostat or other suitable device (not shown) capable of delivering a suitable electrical potential. According to at least one known method, the needle electrode assembly 104 and extending needle 108 for a working electrode that is used in conjunction with another electrode (not shown) separately applied to the skin surface of the subject. In operation, an electrochemical cell is created with cathodic current from the applied working electrode causing a disruption of biofilms on the surface of the implanted orthopedic device. Details relating to the treatment process are found in U.S. Pat. No. 9,616,142, previously incorporated by reference in its entirety.

When the treatment to the metallic implant has been completed, the housing cover 140 can be released by the retaining cap 200. This release permits relative movement of the housing cover 140 within the defined pathway of the housing base 120, including the contained needle electrode assembly 104, and enables retraction of the extending needle 108 and safe encapsulated storage.

Referring to FIGS. 20 and 21 and until this point, the retaining cap 200 has been joining the holder base 120 and holder cover 140 of the positioning and supporting apparatus 100 by means of a set of mechanical snap fits. More specifically and according to this embodiment, the retaining cap 200 is mechanically attached to the upper portion 143 of the holder cover 140 by means of a pair of flexible retention members 220, each having a distal clip portion 224 engaged with the underside of a ledge 121 formed at the top of the sidewalls 124 of the holder base 120, with the projecting tabs 149 of the upper portion 143 of the holder cover 140 being sandwiched therebetween, as shown in FIG. 20.

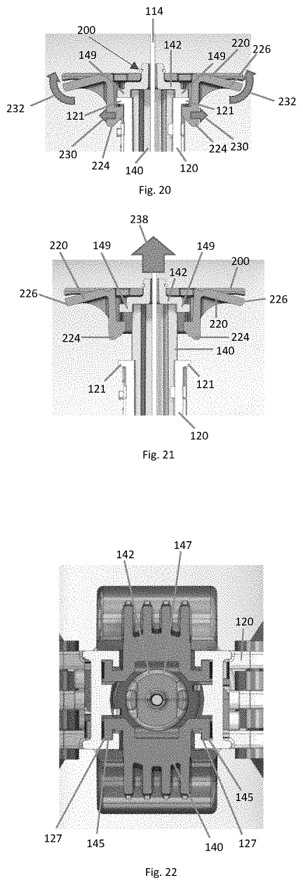

Upon the completion of the treatment to the metallic implant, the user can lift up on a proximal end 226 of the flexible retention members 220, as shown by arrow 232, which causes the distal clip portions 224 to be moved outwardly and out of engagement with the ledge 121 by arrows 230. The preceding action disengages the retaining cap 200 from the holder base 120 and allows the holder cover 140 to move upwardly as a retractable assembly as shown by arrow 238, FIG. 21. This retractable assembly is guided by the holder base 120 through the interlocking channel provided between the holder base 120 and holder cover 140, with the internal slots 127 of the holder base 120 supporting the rail portions 145 of the holder cover 140 and facilitating relative movement, as clearly shown in FIG. 22.

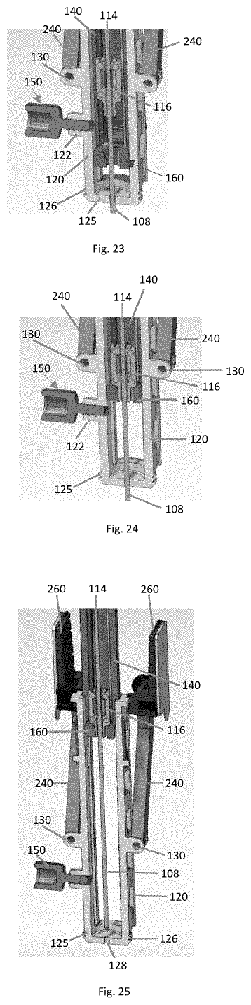

The retractable assembly comprises not only the holder cover 140, but also the retained needle saddle 170, the constant force spring element 160 and the needle electrode assembly 104, each being configured to travel in unison away from the holder base 120 of the herein described apparatus 100 in an upward direction, as shown in FIGS. 23-25. During the upward movement, the needle saddle 160 engages the housing 116 and captures the extending needle 108, pulling the needle electrode assembly 104 from the patient and into the internal cavity or pathway created by the holder base 120. This places the withdrawn needle 108, which would be covered in bacterial material, in an encapsulated state.

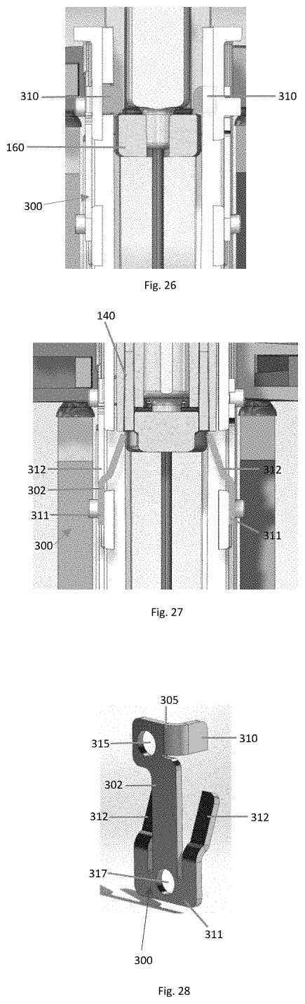

According to this embodiment, upward movement of the needle electrode assembly 104 beyond a predetermined position is prevented by the locking mechanism 300, the latter being disposed proximate the top portion of the holder base 120. With reference to FIGS. 26-28 and according to this specific embodiment, the locking mechanism 300 comprises a pair of substantially planar sections 302, only one being shown and described as shown in FIG. 28 with the remaining section being a mirror image. Each section 302 is preferably made from spring steel and is mechanically attached to a sidewall 124 of the holder base 120 via openings 315 and 317, each of the openings 315 and 317 being sized to engage a nub formed in the sidewall 124. According to this specific embodiment, the planar section 302 of the locking mechanism 300 is mechanically attached to the holder base 120 by at least one heat stake or at least one self-tapping nut, or at least one push-on retaining ring (not shown).

Each section 302 of the locking mechanism 300 according to this embodiment has an upper portion 305 that includes a transverse locking member 310, the latter being configured to extend into the cavity formed by the holder base 120, as well as a lower portion 311 that includes a pair of spaced prongs 312 that protrude on opposite sides of the opening 317. Each prong 312 is configured to extend inwardly and upwardly in spaced relation into the defined cavity of the holder base 120 with the transverse locking member 310 being configured to engage the needle saddle 160 to provide an upper mechanical stop as shown in FIG. 26 and with the prongs 312 being disposed to engage the lower end of the holder cover 140 to prevent downward movement after the needle electrode assembly 104 has been retracted into the apparatus 100. The latter blocking action prevents the needle 108 from protruding from the bottom of the holder base 120 and being redeployed, thereby allowing safe disposal. According to this specific embodiment, each of the sections 302 can be fabricated and formed from a single substantially section of sheet metal, though it will be understood that the locking mechanism 300 can alternatively be made from a plurality of interfitted components, in which other suitable structural materials can be utilized.

### PARTS LIST FOR FIGS. 1-28

100 apparatus
104 needle electrode assembly
108 needle, coated
110 distal end, needle
112 proximal end, needle
114 electrode wire
115 upper section, housing
116 housing, needle electrode assembly
117 electrical connection between electrode wire and needle
118 lower projection, housing
120 holder base
122 projecting portion, holder base
121 ledge, top of sidewalls, holder base
123 open upper end or portion, holder base
124 sidewalls, holder base
125 lower end, holder base
126 threads, lower end of holder base
127 slots, internal, holder base
128 center opening, holder base
129 threaded opening, sidewall
130 support leg mounts
140 holder cover
141 lower recessed portion, holder cover
142 top portion, holder cover
144 open bottom end, holder cover
145 vertical rails, holder cover
146 sidewalls, holder cover
147 upper projecting portion, holder cover
148 center opening, holder cover
149 projecting tabs, top portion
150 needle release member
152 head portion, needle release member
154 shank portion, needle release member
156 threads, shank portion
157 arrow
158 arrow
160 needle saddle
166 center opening, needle saddle
170 spring element
172 ribbon
174 spools or drums, spring element
175 opening, spring element
177 arrows (various spring extensions)
180 protective cap
182 internal cavity
184 upper portion, protective cap
186 threads
188 arrow
189 arrow
200 retaining cover
204 center opening
220 flexible retention members
224 distal clip portion, flexible retention member
226 proximal end, flexible retention member
230 arrow
232 arrow
238 arrow
240 support legs
242 holder base mount end, support leg
244 intermediate leg portion, support leg
246 support foot mount end, support leg
248 ball joint
260 support feet
261 proximal end, support foot
264 opening, proximal end of support foot
265 distal end, support foot
266 base, support foot
272 adhesive pad(s), support foot
274 arrow
276 arrow
278 arrow
300 locking mechanism
302 planar section
305 upper section
310 transverse locking member
311 lower portion
312 locking prongs
315 opening
317 opening

It will be understood that numerous variations and modifications are possible as covered by the following appended claims.

1. In combination, an apparatus used to either support and/or position a needle electrode assembly used to disrupt biofilm from an implanted metallic object, the apparatus comprising:
a body defining a cavity configured to retain the needle electrode assembly, the needle electrode assembly including a needle extending from the body at one end and an opposite end that is couplable to an electrical source via an electrode wire;
a needle adjustment mechanism that enables a length of the needle extending from the body to be adjusted relative to a subject having the implanted metallic object, the needle adjustment mechanism comprising a spring element disposed within the body and in contact with the needle electrode assembly, wherein the spring element is a non-linear spring element that is configured to place the same amount of force onto the needle independent of how much the spring element has traveled.

2. The apparatus according to claim 1, further comprising a plurality of deployable support legs extending from the body, the plurality of deployable support legs being configured for attachment to the skin of a subject and hingably supported by at least one support mount disposed on the body, the plurality of deployable support legs being movable between a stored position above the at least one support mount and a deployed position for placement on the skin of the subject.

3. The apparatus according to claim 2, in which the plurality of deployable support legs are hingably supported to an exterior of the body and are configured for movement between the stored position and the deployed position.

4. The apparatus according to claim 3, including a support foot attached to each of the plurality of deployable support legs, each support foot having an adhesive pad.

5. The apparatus according to claim 4, wherein each of the plurality of deployable support legs further includes a ball joint disposed at one end, each ball joint being configured for attachment with a corresponding support foot.

6. The apparatus according to claim 4, wherein each support foot comprises a base defined by a serpentine configuration, the adhesive pad being attached to an underside of the base.

7. The apparatus according to claim 1, including a cap member releasably attached to the body and configured to cover the length of the needle extending from the body.

8. The apparatus according to claim 7, in which the cap member is threadingly secured to the body.

9. The apparatus according to claim 1, further comprising a retraction mechanism configured to enable the length of the extending needle of the needle electrode assembly to be fully withdrawn into the cavity of the body following treatment.

10. The apparatus according to claim 9, in which the body comprises a holder base retaining a holder cover, the holder cover being releasably attached to the holder base by a retaining cover, wherein release of the retaining cover enables the holder cover to be moved in relation to the holder base to withdraw the extending needle within the body.

11. The apparatus according to claim 10, in which the retaining cover is releasably attached to an upper end of the body and includes at least one releasable clip portion attached to the holder base.

12. The apparatus according to claim 10, wherein the non-linear spring element is attached to a movable lower end of the holder cover.

13. The apparatus according to claim 10, further comprising a locking mechanism configured to retain the needle electrode assembly following withdrawal by the retraction mechanism, including the extending length of the needle, within the body.

14. The apparatus according to claim 13, in which the locking mechanism comprises at least one locking feature extending into the cavity of the body and configured to engage at least one of the needle electrode assembly or the holder cover.

15. The apparatus according to claim 10, including at least one first locking feature that is configured to prevent the needle electrode assembly from being withdrawn through an upper part of the body and at least one second locking feature configured to prevent the needle electrode assembly from being deployed through a lower part of the body.

16. The apparatus according to claim 10, in which one of the holder base and holder cover includes a set of rails and the other of the holder base and holder cover includes a set of internal slots configured to receive the set of rails and permit relative movement.

17. The apparatus according to claim 1, in which the needle electrode assembly is disposed within the body with an entire length of the needle extending therefrom, the first needle adjustment mechanism further including a release member extending into the cavity of the body and configured to prevent adjustment of the needle length.

18. The apparatus according to claim 17, in which the release member is a release knob attached to a sidewall of the body.

19-27. (canceled)

28. An apparatus used to either support and/or position a needle electrode assembly used for disrupting biofilm from an implanted metallic object, the apparatus comprising:
a body defining a cavity sized and configured to retain the needle electrode assembly; and
a needle adjustment mechanism that enables the length of a needle of the needle electrode assembly extending from the body to be adjusted relative to a subject having the implanted metallic object, the needle adjustment mechanism comprising a non-linear spring element supported on a movable portion of the body and configured to apply roughly the same amount of force onto the needle independent of how much the spring element has traveled.
